(19) United States
(12) Patent Application Publication
(10) Pub. No.: US 2020/0352759 A1
(43) Pub. Date: Nov. 12, 2020

(54) RETRIEVAL MECHANISM AND DILATION CATHETER

(71) Applicant: Kawasumi Laboratories, Inc., Oita (JP)

(72) Inventors: Yoshio SAKAYA, Tokyo (JP); Tomoaki YOKOTA, Oita (JP); Toshiyasu YUBA, Tokyo (JP); Kazunori MURAKAMI, Tokyo (JP); Naoaki YAMAMOTO, Oita (JP)

(73) Assignee: Kawasumi Laboratories, Inc., Oita (JP)

(21) Appl. No.: 16/966,075

(22) PCT Filed: Feb. 20, 2019
(86) PCT No.: PCT/JP2019/006271

§ 371 Date: Jul. 30, 2020

(30) Foreign Application Priority Data
Feb. 23, 2018 (JP) 2018-030318
Mar. 23, 2018 (JP) 2018-056272

(51) Int. Cl. A61F 2/958

(52) U.S. Cl. CPC A61F 2/958; A61F 2002/9528

(57) ABSTRACT

The purpose of the present invention is to properly accommodate an expandable structure inside a sheath. This retrieval mechanism is designed to retrieve an expandable member (1) from within a blood vessel (V). The retrieval mechanism is provided with a sheath tube (4) and a retrieval member (3) for retrieving the expandable member into the sheath tube. The retrieval member has a retrieval assistance part (32) for assisting in retrieving the expandable member into the sheath tube. The retrieval assistance part can be accommodated in the sheath tube so as to cover the base part of the expandable member.

### TECHNICAL FIELD

The present invention relates to a retrieval mechanism and a dilation catheter.

### BACKGROUND ART

Conventionally, several balloon catheters have been known which use a balloon to expand an intravascular indwelling device, such as a stent or a stent graft, that is placed at a predetermined position inside a blood vessel (e.g., see Patent Document 1).

### PRIOR ART DOCUMENT

### Patent Document

Patent Document 1: Japanese Unexamined Patent Application Publication No. 2013-188309

### SUMMARY OF THE INVENTION

### Technical Problem

In the case of Patent Document 1 or the like, however, the expanded balloon occludes a blood vessel and blocks blood flow. As such, a configuration may be conceived which secures blood flow using wire for the framework of an expansion/contraction member that expands the intravascular indwelling device. However, in this case, while the expansion/contraction member (wire structure) is being retracted into the sheath, the wire or balloon is likely to get caught on the opening end of the sheath, possibly preventing proper retraction of the expansion/contraction member.

The above-mentioned problems may occur depending on, for example, the form (e.g., size, shape, and material) of the balloon or the sheath, even in a configuration that expands an intravascular indwelling device with blocking blood flow.

An object of the present invention is to provide a retrieval mechanism and a dilation catheter that are capable of properly retracting an expandable and contractible structure into a sheath.

### Solution to Problem

A retrieval mechanism of the present invention is a retrieval mechanism to retrieve an expandable and contractible structure from inside a blood vessel. The retrieval mechanism includes a sheath, and a retrieval member configured to retrieve the structure into the sheath, in which the retrieval member includes a retrieval assistant portion that assists in retrieval of the structure into the sheath, in which the retrieval assistant portion is retractable into the sheath while covering the proximal end part of the structure.

Furthermore, a dilation catheter of the present invention is a dilation catheter to expand an intravascular indwelling device in a cylindrical form indwelled at a predetermined position inside a blood vessel. The dilation catheter includes a sheath, an expandable and contractible structure pressing an inner side surface of the intravascular indwelling device radially outward, and a retrieval member configured to retrieve the structure into the sheath, in which the retrieval member has a retrieval assistant portion that assists in retrieval of the structure into the sheath, and in which the retrieval assistant portion is retractable into the sheath while covering a proximal end part of the structure.

### Advantageous Effect of the Invention

The present invention provides proper retraction of an expandable and contractible structure into a sheath.

### BRIEF DESCRIPTION OF THE DRAWINGS

FIG. 1 is a perspective view illustrating an in-use state of the dilation catheter.

FIGS. 2A and 2B illustrate a state of expansion of an expansion/contraction member of the dilation catheter.

FIG. 3A and FIG. 3B illustrate a state of retraction of the expansion/contraction member of the dilation catheter.

FIG. 4 illustrates an example of the structure of a framework member.

FIGS. 5A to 5D are views for description a method of use of the dilation catheter.

FIGS. 6A to 6D are views for description a method of use of the dilation catheter.

FIG. 7 is a view for description another example of a method of connecting a framework member and a shaft member.

### DESCRIPTION OF THE EMBODIMENT

Hereinafter, the embodiments of the present invention are described in detail with reference to the drawings.

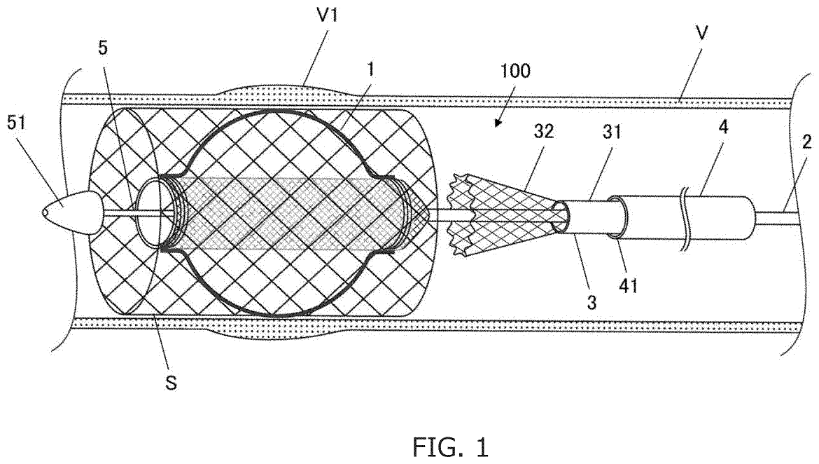

FIG. 1 is a perspective view illustrating an in-use state of a dilation catheter 100 according to an embodiment of the present invention.

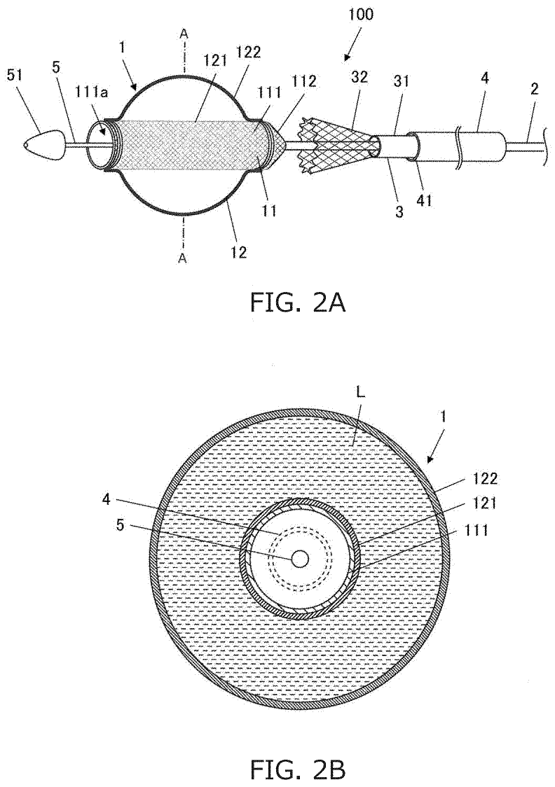

FIG. 1 schematically illustrates an expansion/contraction member 1 of the dilation catheter 100. The same applies to FIGS. 2A and 2B, 5A to 5D, and 6A to 6D described below. In the following description, a farther side (distal side) from a user of the dilation catheter 100 is designated as a tip side, and a closer side (base side) from the user is designated as a proximal end side.

As illustrated in FIG. 1, the dilation catheter 100 expands a cylindrical stent (intravascular indwelling device) S at an indwelling site V1 (e.g., a stenosis site or an occlusion site) inside a blood vessel V.

As the stent S, a known one may be applied which, for example, has a so-called self-expanding configuration provided with storing a shape of an expanded state and is indwelled by a catheter other than the dilation catheter 100, although detailed descriptions is omitted here. The stent S may also be, for example, one that can be introduced into the blood vessel V with being contracted radially inward and attached to the tip of the dilation catheter 100 (an outer peripheral surface of the expansion/contraction member 1).

In addition, the stent S has, for example, a structure with metal element wire woven in a lattice shape, and has a substantially cylindrical outer shape as a whole. Examples of materials of the metal element wires include known metals and metal alloys typified by, for example, Ni—Ti alloy, stainless steel, and titanium alloy. The stent S, for example, is applied with external force radially outward from the inside, thereby expanding radially outward and being indwelled in close contact with the blood vessel.

Here, although the intravascular indwelling device has been exemplified by the stent S, which is merely an example and not limited thereto, it may be arbitrarily changed to a stent graft or the like (illustration omitted) if appropriate.

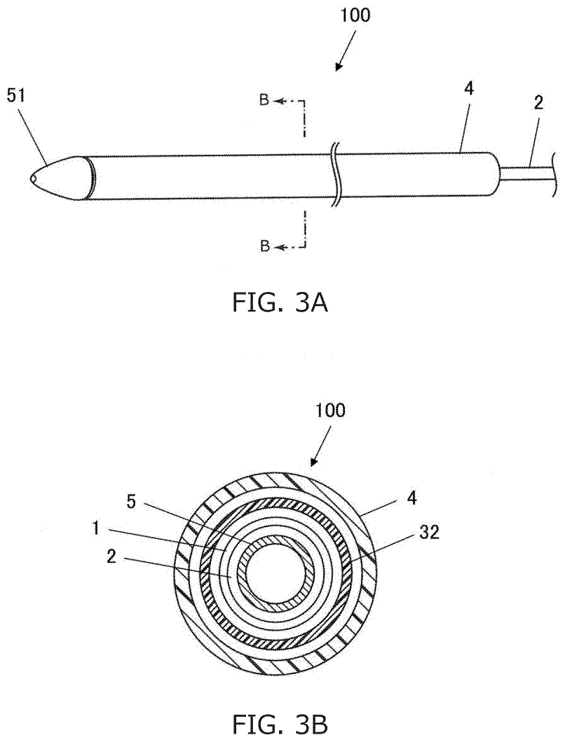

FIGS. 2A and 2B illustrate a state of expansion of the expansion/contraction member 1 of the dilation catheter 100. FIGS. 3A and 3B illustrate a state of retraction of the expansion/contraction member 1 of the dilation catheter 100. Among these, FIGS. 2A and 3A are perspective views of the dilation catheter 100. As well, FIG. 2B is a cross-section view taken along the line AA in FIG. 2A, and FIG. 3B is a sectional view taken along the line BB of FIG. 3A.

The dilation catheter 100 is introduced into the blood vessel V in a retraction state of the expansion/contraction member 1 with the expansion/contraction member 1 being retracted in a sheath tube 4 (see FIGS. 3A and 3B), and is introduced up to the indwelling site of the stent S, followed by exposure of the expansion/contraction member 1 from the sheath tube 4, leading to an expanded state of the expansion/contraction member 1 (see FIGS. 2A and 2B). Furthermore, the expansion/contraction member 1 is configured to be expandable and contractible without blocking blood flow in the blood vessel V, and the dilation catheter 100 includes a retrieval mechanism that retrieves the expansion/contraction member 1 from inside the blood vessel V.

As illustrated in FIGS. 2A and 2B as well as FIGS. 3A and 3B, the dilation catheter 100 includes the expansion/contraction member 1, a balloon tube 2, a retrieval member 3, a sheath tube 4, a guide tube 5, and the like.

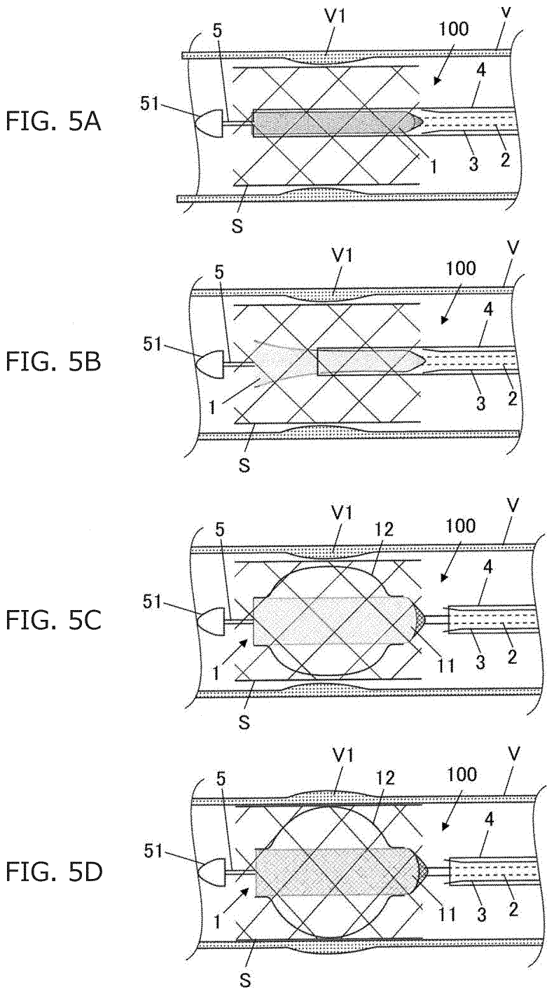

The expansion/contraction member 1 is arranged inside the stent S, and presses the stent S radially outward from the inside via expansion of a balloon 12 (detailed below). The expansion/contraction member 1 is a radially expandable and contractible member, and forms a substantially spherical shape in an expanded state (see FIG. 1 etc.) as well as forms a substantially cylindrical shape in a contracted state (see FIGS. 5A and 5B). The outer diameter of the expansion/contraction member 1 in the contracted state is almost equal to the inner diameter of the sheath tube 4. The expansion/contraction member 1 is reduced in diameter by, for example, radial compression or folding, and is retracted in the sheath tube 4. The expansion/contraction member 1 is attached to the tip part of the balloon tube 2.

Specifically, the expansion/contraction member 1 has a framework member 11 that forms a framework of the expansion/contraction member 1 with wire, and a balloon 12 that is expandable, contractible and deformable.

The balloon 12 is affixed to the framework member 11 so as to cover an outer peripheral surface of the framework member 11. The framework member 11 and the balloon 12 are retracted inside the sheath tube 4 in introduction of the dilation catheter 100 into the blood vessel V (see FIGS. 3A and 3B), and are exposed from the sheath tube 4 in expansion of the balloon 12 (see FIGS. 2A and 2B).

The balloon 12 is an expanded deformation member that expands and uniformly presses the stent S circumferentially. The balloon 12 is also formed of an elastic resin material such as thermoplastic synthetic resin, and for example, has a two-layer structure consisting of an inner layer 121 and an outer layer 122.

The inner layer 121 is formed in close contact with an outer peripheral surface of a cylindrical portion 111 (described below) of the framework member 11 so as to cover it over. In particular, for example, when the cylindrical portion 111 is formed of woven wire, the inner layer 121 is in close contact with the stitches of the wire having spacing (gaps in which the framework wires have spacing) in an expanded state on the outer peripheral surface of the cylindrical portion 111 so as to occlude them.

The outer layer 122 is adhered to the outer peripheral surface of the inner layer 121 at the tip part and the proximal end part. By injecting expansion solution L between the inner layer 121 and the outer layer 122, the outer layer 122 is expanded radially outward in a substantially spherical shape (see FIGS. 2A and 2B).

Here, a shape of the balloon 12 (outer layer 122) in an expanded state has been exemplified with a substantially spherical shape, which is merely an example and not limited thereto, and can be arbitrarily changed as appropriate.

The framework member 11 has the cylindrical portion 111 being in close contact with the balloon 12, and a connecting portion 112 connected to the tip of the balloon tube 2 (shaft member), and has communication between a distal end part and a base end part (both axial ends). As such, even if the balloon 12 expands, blood can flow through a lumen formed by the framework member 11, and the blood flow is not be blocked. Additionally, the balloon 12 is arranged outside the framework member 11, which has a high strength, thereby enabling to provide a pressing force efficiently to the stent S.

The connecting portion 112 is connected to the balloon tube 2 by, for example, suturing with thread, but such connecting method is an example and is not limited thereto, and can be arbitrarily changed as appropriate.

The framework member 11 is also preferably configured to be freely expandable and contractible, and to be in an expanded state as the balloon 12 gets expanded. In particular, the framework member 11 has so-called self-expandability, with storing a shape of an expanded state, and expands radially outward along with being exposed from the sheath tube 4. This provides a smaller expansion volume of the balloon 12 in pressing the stent S against the blood vessel V, and leads to a less volume of the expansion solution L to be injected into the balloon 12, thus allowing a desired pressing force to be easily applied to the stent S.

In addition, the tip part of the cylindrical portion 111 of the framework member 11 may be connected to the guide tube 5 or a chip 51 (described below) to produce a configuration capable of adjusting the expansion volume of the framework member 11 by axially moving the guide tube 5.

Furthermore, the framework member 11 is formed of woven wire, thereby being freely expandable and contractible. The framework member 11 also includes the tip side (distal end side) of the cylindrical portion 111 being open (an opening 111a), and the connecting portion 112 being exposed from the balloon 12. Therefore, blood flows into a lumen of the expansion/contraction member 1 through the opening 111a or a mesh (reference sign omitted) of the connecting portion 112, and flows out into the blood vessel V through the mesh (reference numeral omitted) of the connecting portion 112 or the opening 111a.

Examples of materials of the wire forming the framework member 11 include known metals or metal alloys typified by Ni—Ti alloy, stainless steel, titanium alloy, and the like. Alloy materials having X-ray contrast property may also be used. In this case, the position of the expansion/contraction member 1 can be determined from outside the body.

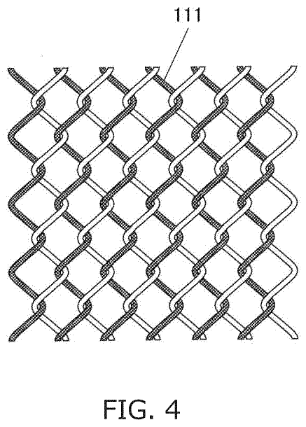

The method of weaving wire is not particularly limited, but for example, a method of weaving a plurality of wires so as to alternately interlock (see FIG. 4) or a method of spirally weaving a plurality of wires (illustration omitted) can be applied. In addition, the cylindrical portion 111 and the connecting portion 112 may be made by different weaving methods.

Then, in the framework member 11, at least the cylindrical portion 111 preferably has a configuration that provides little axial extension. For example, when the framework member 11 is formed by the method of weaving a plurality of wires so as to alternately interlock them as illustrated in FIG. 4, even if it is axially pulled, axial deformation (extension) is controlled by adjoining wires, thereby providing a smaller amount of axial elongation compared to that formed by the method of spirally weaving a plurality of wires.

Moreover, since the framework member 11 has the configuration, which provides little axial extension, and thereby increases in radial rigidity, the lumen of the framework member 11 is less likely to be collapsed in expansion of the balloon 12, thus allowing the blood flow path to be securely retained. In addition, the expansion/contraction member 1 undergoes so small axial deformation during its transitions from a contracted state to an expanded state, that the expansion/contraction member 1 can be easily positioned at a desired indwelling site.

Furthermore, little axial elongation of the framework member 11 leads to little axial extension of the entire expansion/contraction member 1, for example, the expansion/contraction member 1 can be reduced in diameter and retracted into the sheath tube 4 with little axial elongation.

As described above, the expansion/contraction member 1 constitutes an expandable and contractible structure that presses the inner surface of the stent (intravascular indwelling device) S radially outward.

The radially inside of the balloon tube 2 includes the guide tube 5 arranged therein.

The guide tube 5 is one through which a guide wire (illustration omitted) is introduced when the dilation catheter 100 is introduced into a blood vessel. In addition, a chip 51 is attached at the tip of the guide tube 5.

The chip 51 has, for example, a shape in which the outer diameter on the proximal end side is substantially the same as the inner diameter of the sheath tube 4 and decreases in size toward the tip side.

The radially outside of the balloon tube 2 includes the retrieval member 3 arranged thereon.

The retrieval member 3 is for retrieving the expansion/contraction member 1 into the sheath tube 4. Specifically, the retrieval member 3 has a long tubular portion 31 inserted into the sheath tube 4, and is consecutively provided, at the tip part, with a retrieval assistant portion 32 that assists retrieval of the expansion/contraction member 1 into the sheath tube 4.

The retrieval assistant portion 32 is configured to be radially expandable and contractible, in an expanded state, it can introduce the proximal end part (base end part) of the expansion/contraction member 1, e.g., the connecting portion 112 of the expansion/contraction member 1 and the outer layer 122 of the balloon 12, from the tip side, and dispose it inside. Specifically, the retrieval assistant portion 32 is open at the tip side as well as expands the diameter so as to be capable of covering the proximal end part of the expansion/contraction member 1. That is, the outer diameter of the tip part in an expanded-diameter state of the retrieval assistant portion 32 is larger than the outer diameter of the proximal end part in a contracted state of the expansion/contraction member 1. The retrieval assistant portion 32 is also formed in a taper shape which is reduced in the diameter toward the proximal end side from the tip side to the proximal end side in an expanded state.

Moreover, the retrieval assistant portion 32 is configured to be contractible so as to provide the outer diameter of the retrieval assistant portion 32 with a size smaller than that of the inner diameter of the sheath tube 4 when retracted into the sheath tube 4. Specifically, when the retrieval assistant portion 32 is retracted into the sheath tube 4, the outer surface of the retrieval assistant portion 32 comes into contact with the opening end 41 of the sheath tube 4 to let a force be applied radially inward to the outer surface of the retrieval assistant portion 32, thereby making the retrieval assistant portion 32 radially contractible. Then, in response to the contraction of the retrieval assistant portion 32, a force is applied radially inward to the outer surface of the proximal end part of the expansion/contraction member 1 disposed inside the retrieval assistant portion 32, thus making the proximal end part of the expansion/contraction member 1 radially contractible.

In this way, the retrieval assistant portion 32 is configured to cover the proximal end part of the expansion/contraction member 1, and is configured to contract so as to provide the outer diameter with a size smaller than that of the inner diameter of the sheath tube 4, thereby allowing to be retracted into the sheath tube 4.

Furthermore, the retrieval assistant portion 32 is formed of, for example, woven wire, and is freely expandable and contractible. In particular, the retrieval assistant portion 32 has so-called self-expandability with storing a shape of an expanded state, and expands radially outward toward the tip side along with being exposed from the sheath tube 4.

The spaces between the wires composing the retrieval assistant portion 32 are preferably smaller than the spaces between wires of the framework member 11 so that the connecting portion 112 of the framework member 11 is less likely to get caught.

In addition, examples of materials of the wires forming the retrieval assistant portion 32 include known metals or metal alloys typified by Ni—Ti alloy, stainless steel, titanium alloy, and the like. Alloy materials having X-ray contrast property may also be used.

The radially outside of the retrieval member 3 includes the sheath tube 4 arranged thereon.

In other words, the sheath tube 4, the tubular portion 31 of the retrieval member 3, the balloon tube 2, and the guide tube 5 are arranged in a nested order from the radially outer side. The sheath tube 4, the retrieval member 3 (the tubular portion 31), the balloon tube 2, and the guide tube 5 can also be axially moved independently of each other.

Additionally, each of the sheath tube 4, the tubular portion 31 of the retrieval member 3, the balloon tube 2, and the guide tube 5 is, for example, a long tubular member formed of flexible material. Examples of the flexible material include synthetic resin (elastomer), a resin compound in which other materials are mixed with synthetic resin, a multi-layer structure composed of multiple layers of synthetic resin, or a composite of synthetic resin and metal wire.

Incidentally, although illustration is omitted, the dilation catheter 100 may have an operation unit operated by the user, on the proximal end side. In addition, a fluid injection tube (illustration omitted) for injecting an expansion solution (e.g., physiological saline) L or gas into the balloon 12 is inserted into the sheath tube 4, the retrieval member 3, or the balloon tube 2. The tip of the fluid injection tube is inserted inside the balloon 12 (between the inner layer 121 and the outer layer 122).

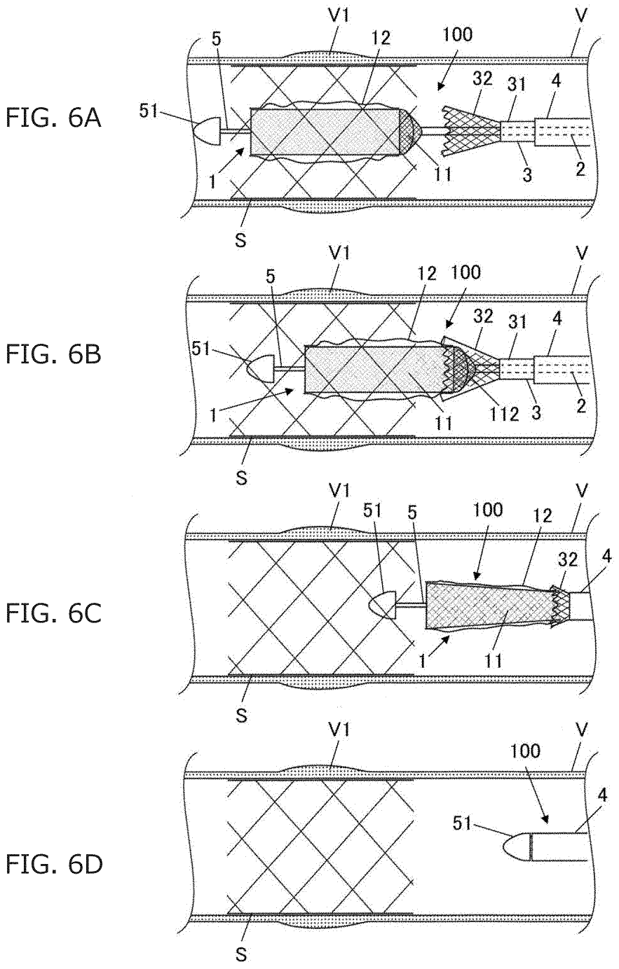

Next, a method of use of the dilation catheter 100 is described with reference to FIGS. 5A to 5D and FIGS. 6A to 6D.

In the following description, it is assumed that a stent S with an insufficient expansion volume is indwelled at a predetermined position V1 (e.g, a stenosis site) inside the blood vessel V. It is also assumed that a guide wire (illustration omitted) is inserted through the blood vessel V beforehand, and that the dilation catheter 100 is to be introduced along this guide wire.

First, a method of use in expanding the stent S using the dilation catheter 100 is described with reference to FIGS. 5A to 5D.

FIGS. 5A to 5D are views for description a method of use of the dilation catheter 100, and schematically illustrate the state in which the stent S is expanding.

As illustrated in FIG. 5A, first, the dilation catheter 100 is inserted into the blood vessel V along the guide wire (illustration omitted) inserted into the blood vessel V, and the expansion/contraction member 1 is positioned inside the stent S.

Next, as illustrated in FIGS. 5B and 5C, the sheath tube 4 is moved axially toward the proximal side (hand side) with the expansion/contraction member 1 being held in place, thereby releasing the expansion/contraction member 1 into the blood vessel V from the sheath tube 4. The part of the expansion/contraction member 1 exposed from the sheath tube 4 expands with self-expanding force of the framework member 11. At this time, the balloon 12 also elastically deforms following the expansion of the framework member 11.

Here, the expansion/contraction member 1 may also be released from the sheath tube 4 by moving the expansion/contraction member 1 along the axial direction so as to push it out toward the distal side (tip side), with the sheath tube 4 being held in place.

At this state, upon injection of the expansion solution L into the balloon 12, the outer layer 122 of the balloon 12 is inflated radially outward and comes into contact with the inner peripheral surface of the stent S. In such state, the tip part of the expansion/contraction member 1 (the opening 111a of the cylindrical portion 111 of the framework member 11) and the proximal end (the mesh of the connecting portion 112 of the framework member 11 (reference numeral omitted)) are in communication, and thus the expansion/contraction member 1 secures blood flow in the blood vessel V.

As the expansion/contraction member 1 further comes to expand, the inner surface part of the blood vessel V becomes pressed radially outward, so that the stenosis site V1 gets expanding (see FIG. 5D). Even in such state, blood flow of the blood vessel V is secured.

Next, a method of use in retrieving the expansion/contraction member 1 into the sheath tube 4 is described with reference to FIGS. 6A to 6D.

FIGS. 6A to 6D are views for description a method of use of the dilation catheter 100, and schematically illustrate a state in which the expansion/contraction member 1 is being retrieved into the sheath tube 4.

As illustrated in FIG. 6A, first, the expansion solution L in the balloon 12 is discharged to shrink the balloon 12, and the retrieval member 3 is exposed from the sheath tube 4 such that the retrieval assistant portion 32 is arranged on the proximal end side of the expansion/contraction member 1. The part of the retrieval assistant portion 32 exposed from the sheath tube 4 expands with self-expanding force of the retrieval assistant portion 32. In an expanded state of the retrieval assistant portion 32, the outer diameter of its tip side is larger than the outer diameter of the contracted expansion/contraction member 1.

Next, as illustrated in FIG. 6B, for example, the balloon tube 2 is moved toward the proximal side (hand side) along the axial direction, with the sheath tube 4 and the retrieval member 3 being held in place. This allows the proximal end part of the expansion/contraction member 1 (e.g., the connecting portion 112 of the expansion/contraction member 1 and the outer layer 122 of the balloon 12) to be inserted into and disposed inside the retrieval assistant portion 32 from its tip side.

Subsequently, as illustrated in FIG. 6C, the retrieval member 3 and the balloon tube 2 are moved toward the proximal side (hand side) along the axial direction relative to the sheath tube 4, thereby allowing the outer surface of the retrieval assistant portion 32 to come into contact with the opening end 41 of the sheath tube 4 to let a force be applied radially inward to the outer surface of the retrieval assistant portion 32. Then, as the retrieval assistant portion 32 comes to contract radially, the proximal end part of the expansion/contraction member 1 disposed inside the retrieval assistant portion 32 also similarly comes to contract radially, so that the expansion/contraction member 1 becomes retrieved into the sheath tube 4.

Upon retrieval of the entire expansion/contraction member 1 into the sheath tube 4 (see FIG. 6D), the dilation catheter 100 is pulled out from the blood vessel V while the stent S is left indwelled.

As described above, the dilation catheter 100 according to the present embodiments is the dilation catheter 100 that expands the cylindrical stent S (intravascular indwelling device) indwelled at a predetermined position inside the blood vessel V, includes the sheath tube 4, the expandable and contractible expansion/contraction member 1 (structure) that presses the inner surface of the stent S radially outward, and the retrieval member 3 for retrieving the expansion/contraction member 1 into the sheath tube 4, in which the retrieval member 3 has the retrieval assistant portion 32 that assists retrieval of the expansion/contraction member 1 into the sheath tube 4, and wherein the retrieval assistant portion 32 is configured to be capable of covering the proximal end part of the expansion/contraction member 1 and being retracted into the sheath tube 4.

Therefore, when the expansion/contraction member 1 is retrieved into the sheath tube 4, the proximal end part of the expansion/contraction member 1 is covered by the retrieval assistant portion 32 of the retrieval member 3, thereby allowing the proximal end part of the expansion/contraction member 1 to be less likely to get caught on the opening end 41 of the sheath tube 4. For example, even when the expansion/contraction member 1 has the framework member 11, the balloon 12, and the like so as not to block blood flow in a state of being indwelled inside the blood vessel V, covering the proximal end part, such as the framework member 11 and the balloon 12, with the retrieval assistant portion 32 allows retraction into the sheath tube 4 without getting caught on the opening end 41 of the sheath tube 4.

In this way, retraction of the expansion/contraction member 1 into the sheath tube 4 can be properly performed.

Furthermore, the retrieval member 3 is consecutively provided with a retrieval assistant portion 32 at the tip part, and further has a tubular portion 31 inserted into the sheath tube 4. The retrieval assistant portion 32 is configured to be expandable and contractible in the radial direction orthogonal to the axial direction of the tubular portion 31, and is capable of inserting the proximal end part of the expansion/contraction member 1 from the tip side and disposing it inside in an expanded state, and is configured to be contractible so as to provide the outer diameter of the retrieval assistant portion 32 with a size smaller than that of the inner diameter of the sheath tube 4 when retracted into the sheath tube 4.

Therefore, the expandable and contractible retrieval assistant portion 32 can cover the proximal end part of the expansion/contraction member 1 to make the proximal end part of the expansion/contraction member 1 less likely to get caught on the opening end 41 of the sheath tube 4 in an expanded state, and can properly retract it into the sheath tube 4 in a contracted state.

Particularly, when the retrieval assistant portion 32 is retracted into the sheath tube 4, the outer surface of the retrieval assistant portion 32 comes into contact with the opening end 41 of the sheath tube 4 to let a force be applied radially inward to the outer surface of the retrieval assistant portion 32, thereby allowing the retrieval assistant portion 32 to be contracted. Furthermore, in response to the contraction of the retrieval assistant portion 32, the proximal end part of the expansion/contraction member 1 disposed inside the retrieval assistant portion 32 can be contracted radially, thereby allowing the expansion/contraction member 1 to be easily retracted into the sheath tube 4. This enables to perform a retrieval operation of the expansion/contraction member 1 from inside the blood vessel V efficiently in a short time.

Hereinbefore, the invention made by the present inventor has been specifically described based on the embodiments, but the present invention is not limited to the above embodiments and can be modified without departing from the scope.

For example, the configuration of the retrieval assistant portion 32 illustrated in the above embodiment is an example and is not limited thereto, it may have larger spaces between wires composing the retrieval assistant portion 32 than the spaces between wires of the framework member 11, or may be a configuration provided with a film body so as to occlude the gap between wires (illustration omitted). A retrieval assistant portion (illustration omitted) may also be configured by flare-processing the tip part of the tubular portion 31 of the retrieval member 3.

Moreover, in the above embodiment, the expansion/contraction member 1 has been illustrated as being composed of the framework member 11, the balloon 12, and the like, so as not to block blood flow in the blood vessel V. However, it is an example and is not limited thereto, and for example, it may be configured to block blood flow (e.g., a balloon (illustration omitted)).

The expansion/contraction member 1 also has been illustrated in the case of expanding in a substantially spherical shape, but it is an example and is not limited thereto, and the shape of the expansion/contraction member 1 in an expanded state can be arbitrarily changed as appropriate. In other words, any shape may be used so long as the expansion/contraction member 1 in an expanded deformed state can press the intravascular indwelling device, such as the stent S, radially outward uniformly.

Furthermore, although not particularly specified in the embodiments, the outer layer 122 may be extended relatively easily compared to the inner layer 121. In particular, the inner layer 121 and the outer layer 122 may have difference in, for example, at least one of thickness and material, thereby rendering the outer layer 122 extended relatively easily compared to the inner layer 121. In other words, the outer layer 122 may be made extended relatively easily compared to the inner layer 121 by providing the thickness of the inner layer 121 larger than the thickness of outer layer 122 using the same materials, or providing the inner layer 121 and the outer layer 122 with the same thickness and forming them with resin materials that produce the inner layer 121 harder than the outer layer 122, or providing the thickness of the inner layer 121 larger than the thickness of the outer layer 122 and forming them with resin materials that produce the inner layer 121 harder than the outer layer 122, or the like.

In such case, the inner layer 121 of the balloon 12 is less likely to be expanded and deformed, while the outer layer 122 is more likely to be expanded and deformed radially outward. Thus, in expansion and deformation of the balloon 12, while the cylindrical portion 111 is suppressed not to be crushed inward to cause blocking of the blood flow, the expansion of balloon 12 can provides, for example, proper expansion of the stent S.

In addition, it is desirable that the outer layer 122 of the balloon 12 be easily expanded and deformed while having a predetermined strength. That is, by having, for example, a predetermined thickness and hardness, the outer layer 122 can be less likely to tear even in contact with the inside of the retrieval assistant portion 32 during retrieval of the expansion/contraction member 1, and can perform retrieval of the expansion/contraction member 1 more properly. In view of improving the strength of the outer layer 122, a configuration may also be made of a plurality of two or more layers, in which the inner side may be an expandable and deformable layer and the outer side may be a layer having a predetermined strength.

Incidentally, in the embodiments, although a case has been described where the connecting portion 112 provided on the proximal end side of the cylindrical portion 111 is connected to the balloon tube 2 (shaft member), other structures may also be applied.

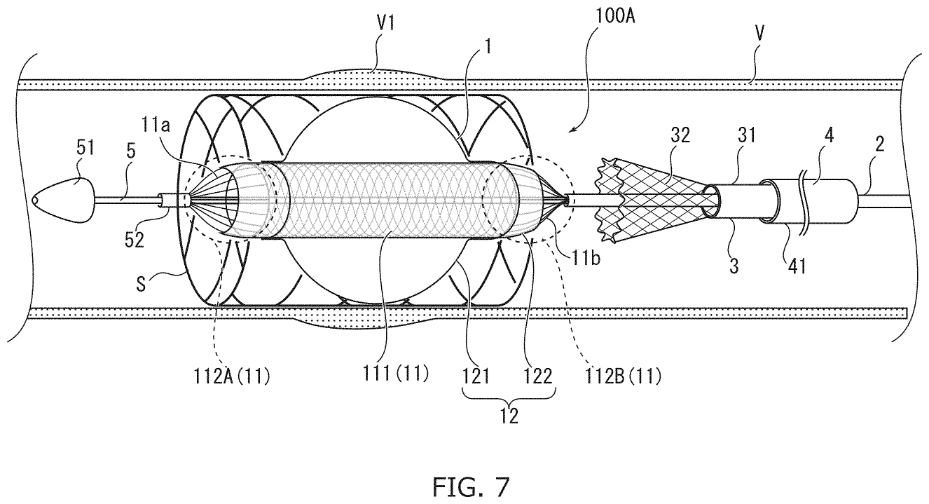

For example, as can be seen with the dilation catheter 100A illustrated in FIG. 7, a tip-side connecting portion 112A and a proximal-end-side connecting portion 112B (parts surrounded by broken lines) may be provided on the tip side and proximal end side of the cylindrical portion 111, respectively, so that the tip-side connecting portion 112A and the proximal-end-side connecting portion 112B may be connected to the holding tube 52 (shaft member) and the balloon tube 2 (shaft member), respectively.

Specifically, the tip-side connecting portion 112A has a configuration in which a plurality of wires 11a extending from the tip part of the cylindrical portion 111 toward the tip side are converged at the center and connected to the holding tube 52. The holding tube 52 receives insertion of the guide tube 5 thereinto and is held so as to be axially movable. Each of the wires 11a of the tip-side connecting portion 112A are also arranged at intervals in the circumferential direction of the holding tube 52, and connected to the holding tube 52 so as to form a radial pattern.

In contrast, the proximal-end-side connection part 112B has a configuration in which a plurality of wires lib extending from the proximal end of the cylindrical portion 111 toward the proximal end side are converged at the center and connected to the balloon tube 2. Each of the wires 11b of the proximal-end-side connection portion 112B are arranged at intervals in the circumferential direction of the balloon tube 2, and connected to the balloon tube 2 so as to form in a radial pattern.

Moreover, the tip side of the inner layer 121 reaches a position covering a part of the wires 11a of the tip-side connecting portion 112A, and the proximal end side of the inner layer 121 reaches a position covering a part of the wires lib of the proximal-end-side connecting portion 112B. Consequently, the overall shape of the inner layer 121 forms a cylindrical shape in which both ends on the tip side and the proximal end side are slightly narrowed.

In this way, by providing the tip-side connecting portion 112A and the proximal-end-side connecting portion 112B at both ends of the cylindrical portion 111, the framework member 11 is stably held substantially coaxially with the balloon tube 2 and the guide tube 5. In addition, this can improve the radial rigidity of the framework member 11, and the lumen of the framework member 11 becomes less likely to be collapsed in expansion of the balloon 12, thus allowing the blood flow path to be securely retained.

Here, the shaft member does not have to be a tube (pipe) shape, but may be a wire (linear) shape. The tip-side connecting portion 112A and the proximal-end-side connecting portion 112B may also be connected to the same shaft member.

The framework member 11 may also be formed, for example, by laser processing (laser cutting) of a single metal pipe (e.g., a pipe made of a Ni—Ti alloy).

Furthermore, a stent expansion apparatus may be formed having a configuration in which the stent S or a stent graft contracted radially inward is attached to the tip side of the dilation catheter 100, thereby allowing the stent S or the stent graft to be indwelled at a predetermined position inside the blood vessel V more easily.

Additionally, in the present invention, any configuration may be employed as long as it is a retrieval mechanism that retrieves the expandable and contractible expansion/contraction member 1 from inside the blood vessel V. In particular, the configuration only needs to include the sheath tube 4 and the retrieval member 3 for retrieving the expansion/contraction member 1 into the sheath tube 4, in which the retrieval member 3 has the retrieval assistant portion 32 that assists retrieval of the expandable and contractible expansion/contraction member 1 into the sheath tube 4, and wherein the retrieval assistant portion 32 is configured to be capable of covering the proximal end part of the expansion/contraction member 1 and being retracted into the sheath tube 4.

It should be understood that the embodiments disclosed herein are illustrative in all respects and are not restrictive. The scope of the present invention is indicated not by the above description but by the claims, and it is intended to encompass all modifications within the spirit and scope equivalent to the claims.

The contents of disclosures of the specifications, drawings, and abstracts in Japanese Patent Application No. 2018-30318 filed on Feb. 23, 2018 and Japanese Patent Application No. 2018-56272 filed on Mar. 23, 2018 are incorporated herein in their entirety.

### DESCRIPTION OF REFERENCE NUMERALS

100 Dilation catheter
1 Expandable and contractible member (structure)
11 Framework member
111 Cylindrical portion
12 Balloon
3 Retrieval member
31 Tubular portion
32 Retrieval assistant portion
4 Sheath tube
41 Opening end
S Stent (intravascular indwelling device)
V Blood vessel

1. A retrieval mechanism to retrieve a structure that is expandable and contractible from inside a blood vessel, comprising:
a sheath; and
a retrieval member configured to retrieve the structure into the sheath,
wherein the retrieval member includes a retrieval assistant portion that assists in retrieval of the structure into the sheath, the retrieval assistant portion being retractable into the sheath while covering a proximal end part of the structure.

2. The retrieval mechanism according to claim 1, wherein
the retrieval member further includes a tubular portion inserted into the sheath, the tubular portion including a tip part where the retrieval assistant portion is provided consecutively, and
the retrieval assistant portion is expandable and contractible in a radial direction orthogonal to an axial direction of the tubular portion, allows, in an expanded state, the proximal end part of the structure to be inserted from a tip side into the retrieval assistant portion and disposed inside, and is contractible to have an outer diameter with a size smaller than a size of an inner diameter of the sheath when retracted into the sheath.

3. The retrieval mechanism according to claim 2, wherein, when the retrieval assistant portion is retracted into the sheath, an outer surface of the retrieval assistant portion comes into contact with an opening end of the sheath to let force be applied radially inward to the outer surface of the retrieval assistant portion, thereby making the retrieval assistant portion contracted.

4. The retrieval mechanism according to claim 2, wherein, in response to contraction of the retrieval assistant portion, the proximal end part of the structure disposed inside the retrieval assistant portion is radially contractible.

5. The retrieval mechanism according to claim 1, wherein the retrieval assistant portion is formed in a tapered shape having a diameter decreased in size toward a proximal end side.

6. The retrieval mechanism according to claim 1, wherein
the structure includes a framework formed of wires, and
the retrieval assistant portion is formed by weaving wires and has spaces smaller than spaces between the wires of the structure.

7. A dilation catheter to expand an intravascular indwelling device in a cylindrical form indwelled at a predetermined position inside a blood vessel, comprising:
a sheath;
a structure that is expandable and contractible, and presses an inner surface of the intravascular indwelling device radially outward; and
a retrieval member configured to retrieve the structure into the sheath,
wherein the retrieval member includes a retrieval assistant portion that assists in retrieval of the structure into the sheath, the retrieval assistant portion being retractable into the sheath while covering a proximal end part of the structure.

8. The dilation catheter according to claim 7, wherein the structure includes:
a framework member including a cylindrical portion and axial end parts, the axial end parts communicating with each other; and
a balloon that is arranged outside the cylindrical portion and expanded by supply of a fluid to press the inner surface of the intravascular indwelling device radially outward,
and allows an unblocked blood flow in a state of being indwelled inside the blood vessel.

9. The dilation catheter according to claim 8, wherein
the balloon includes an inner layer in close contact with an outer peripheral surface of the cylindrical portion, and an outer layer arranged outside the inner layer, the outer layer being made to extend relatively easily compared to the inner layer, and
the outer layer is expanded and deformed radially outward by supplying a fluid between the inner layer and the outer layer in an expanded state of the cylindrical portion.

10. The dilation catheter according to claim 7, wherein the framework member further includes a connecting portion formed at an axial end part of the cylindrical portion, the connecting portion being connected to a shaft member that disposes the structure inside the intravascular indwelling device.
